(19) United States
(12) Patent Application Publication
(10) Pub. No.: US 2007/0191676 A1
(43) Pub. Date: Aug. 16, 2007

(54) UROLOGICAL RESECTOSCOPE STEM

(75) Inventor: Pieter Brommersma, Bargteheide (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/672,248

(22) Filed: Feb. 7, 2007

(30) Foreign Application Priority Data
Feb. 14, 2006 (DE) 102006006950.1

(51) Int. Cl. A61B 1/00; A61B 1/12

(52) U.S. Cl. 600/105; 600/156; 600/129

(57) ABSTRACT

A urological resectoscope stem (1) including an insulating muff (11) constituting the end zone of the stem (1), further including an outer tube (2, 9), an inner tube (3) and a cross-sectionally annular distally closed return flow duct (4) which is subtended at least over the larger part of its length between the outer tube (2, 9) and the inner tube (3), where the stem (1) is fitted with outwardly open, distally extending grooves (10), wherein the annular return flow duct (4) communicates directly by apertures (19) with the grooves (10).

### BACKGROUND OF THE INVENTION

Urological resectoscopes are inserted by means of their stem through the urethra as far as into the area of surgery, which as a rule is in the prostate. Using an hf-loaded loop, resection is carried out in front of the distal stem end toward the insulating muff. An optics configured within the stem allows observation of the area of surgery. To foremost eliminate clouding caused by bleeding, modern resectoscopes operate with constant rinsing. Rinsing liquid is applied through the inner tube and drained through the return duct between the inner and outer tubes. The flow from outside into inside the return duct takes place through outward pointing apertures in the return duct.

The stem being firmly enclosed by the urethra from its proximal zone far toward its distal end apertures that are configured more proximally are sealed off and the return flow then only takes place through the distal apertures. As a result, the design of conventional resectoscope stems emphasizes configuring the apertures as distally as possible.

The design of the German patent document DE 76 26 244 U comprises an axial internal groove covered by an inner tube tongue and fitted with a row of outward pointing apertures, This configuration makes it possible to fit apertures at least along a longitudinal line distally away from the end of the outer tube into the insulating muff. However such a design is complex and provides distal apertures only along a line parallel to the axis.

The related U.S. Pat. document No. 6,712,759 B2 provides several circumferentially spaced, axial ducts in the insulating muff's proximal zone of reduced outside diameter which is overlapped by the outer tube, these ducts being covered by the outer tube and connected to the cross-sectionally circular and otherwise distally sealed return duct. Said ducts distally project beyond the outer tube end and constitute in that zone outwardly open grooves which communicate via said ducts with the return duct. Said apertures are advantageously situated very distally; on the other hand the overall design is complex. Such a design leads to very narrow ducts or, as shown by the cited work, the diameter must be substantially enlarged in the zone of the insulating muff because the inner tube, the muff, the return duct and outer tube are superposed in segments, entailing problems in space minimization.

The objective of the present invention is to design a stem of the above kind offering more advantageous spatial and diameter configurations.

### BRIEF SUMMARY OF THE INVENTION

In the present invention, distal apertures of the return flow duct are directly opening out into the stem-configured grooves that are open outward and that extend distally away from the apertures. A liquid can flow through these grooves even if they are covered with body tissue when the proximal end zone of these grooves itself is covered by the body tissue above the apertures. Accordingly the grooves allow a return flow even when body tissue encloses the stem far into the distal zone. The invention offers a very simple stem design whereby in particular also its outside diameter may be minimized because the groove design differs from that of the known U.S. Pat. document No. 6,712,759 B2 wherein such grooves are covered by the outer tube to form the ducts.

The grooves can be constituted in the insulating muff and may even run through this muff as far as its distal edge, as a result of which a return flow shall still be possible when the stem is laterally completely enclosed. Advantageously however, the grooves shall be made in the outer tube, and accordingly they can be manufactured in simple manner by metal-working. In the process, the distal rim of the outer tube shall be closed as a result of which the grooves are completely enclosed by the metal of the outer tube, The closed distal rim assures improved dimensional stability of the outer tube in that zone.

Depending on the manufacturing procedure, the insulating muff may be affixed to the inner tube or preferably to the outer tube, the latter embodiment allowing improved diameter ratios. The insulating muff may be connected in an abutting manner to the distal outer tube edge, for instance by soldering. Advantageously however, the insulating muff is overlapped within a proximal zone of reduced diameter by the outer tube. Due to the attendant overlap soldering or bonding then may be implemented over an enlarged affixation area.

In this embodiment mode, the grooves in the outer tube may run beyond the reduced zone of the insulating muff, that is very far distally. Advantageously, the grooves may be recessed to a depth reaching into the insulating muff's material, thereby subtending a larger cross-section for the flow in them.

As a result the soldering or bonding affixation of the outer tube to the insulating muff may be improved and a good resting surface is assured to the inner tube edge.

The distal end of the groove-fitted outer tube may be integral with the tube's proximal end. However, the distal end of the outer tube is a separate perforated collar which, following its complex manufacture, shall be affixed to the straight proximal portion of the outer tube for instance by welding the metal parts to each other.

### BRIEF DESCRIPTION OF THE DRAWINGS

The drawings elucidate the present invention in illustrative and schematic manner.

FIG. 1 is a top view of the distal end of a first embodiment of a stem of the present invention,

FIG. 2 is a section along line 2-2 in FIG. 1,

FIG. 3 is a section along line 3-3 in FIG. 1,

FIG. 4 is a section corresponding to FIG. 2 of another embodiment mode,

FIG. 5 is a section corresponding to FIG. 2 through another embodiment mode,

FIG. 6 is a section corresponding to FIG. 2 through a further embodiment mode, and

FIG. 7 is a section corresponding to FIG. 2 through still another embodiment mode.

### DETAILED DESCRIPTION OF THE INVENTION

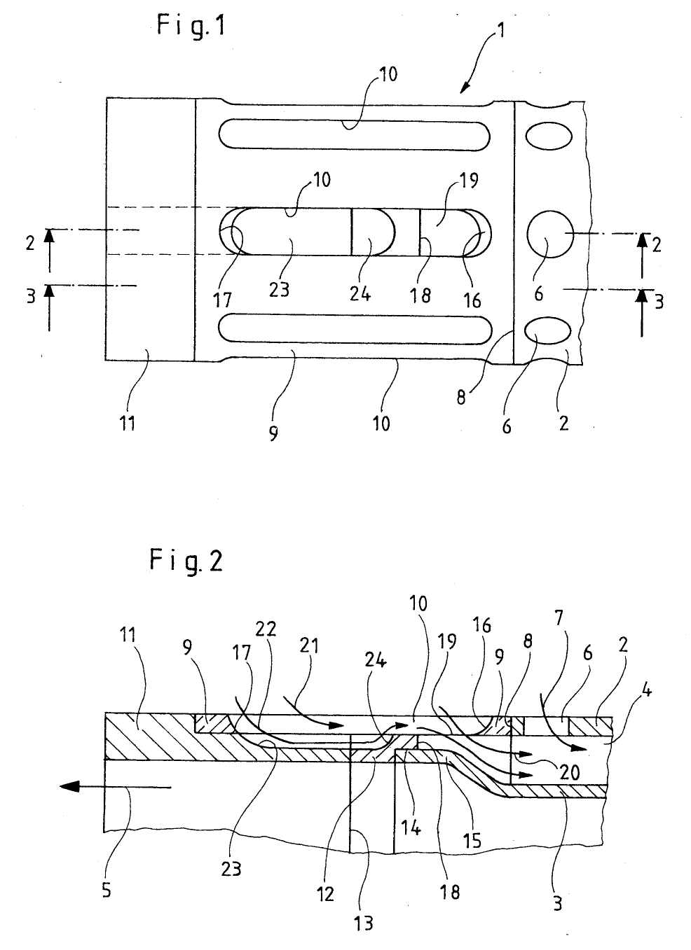

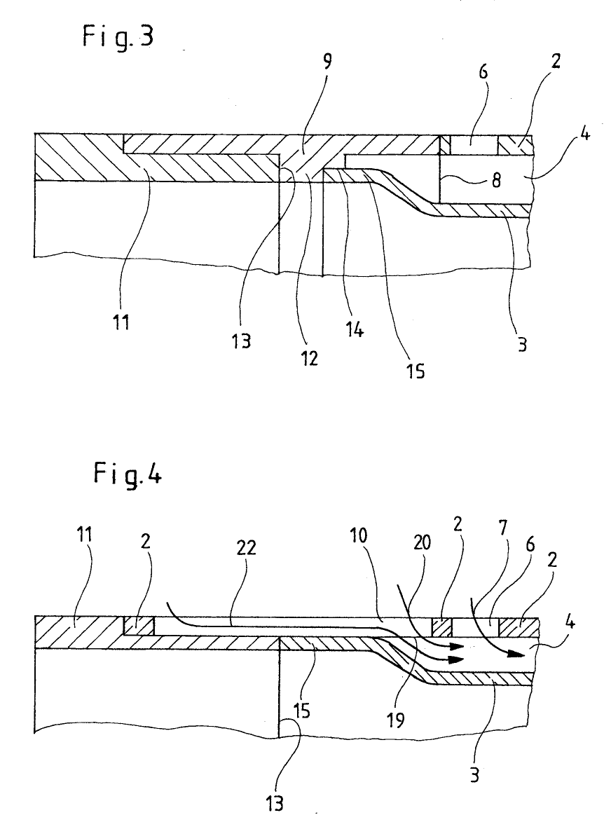

FIG. 1 is a top view of the end of a stem 1 of a urological resectoscope, its proximal zone being omitted. The proximal part of the stem illustratively may be designed in the manner of U.S. Pat. No. 6,712,759. FIGS. 2 and 3 are sections parallel to the axis and through the wall of the stem 1 at different circumferential sites.

FIG. 3 shows that in its longitudinal, proximal main part, said stem comprises an outer tube 2 and an inner tube 3. An annular gap is subtended between said tubes and acts as the return flow duct 4 to feed a liquid to an omitted rinsing fitting in the proximal zone of the stem 1. A liquid flows within the inner tube 3 from an omitted rinsing fitting in the proximal zone of the stem 1 as indicated by the arrow 5 of FIG. 2 toward the surgical area and drains from the latter through the return flow duct 4.

Outwardly open apertures 6 are configured in the distal part of the outer tube 2 at the return flow duct 4 to pass rinsing liquid in the direction of the arrow 7 of FIG. 2 into the return flow duct 4. A perforated collar 9 is affixed, for instance, by a weld seam 8 and, as shown in FIG. 1, comprises several axial distal grooves 10 externally fitted at this site into the stem 11. The perforated collar 9 rests by its distal end which in this embodiment exhibits the same wall thickness as the outer tube 2 on a zone of an insulating muff 11, illustratively of ceramic, and of reduced outside diameter, The perforated collar 9 is affixed, for instance, by area bonding, soldering or similar means on the insulating muff 11.

In the embodiment mode shown in FIGS. 2 and 3, the perforated collar is fitted with an inner flange 12 abutted by the proximal edge 13 of the insulating muff 11 and widened at its proximal zone at the surface 14 to the diameter exhibited by the inner tube 3 which is widened at its distal end zone 15. Accordingly, the inner tube 3 rests in a sealing manner in the zone of the surface 14 against the outer tube 2 assuming the form of a flange 12 which is affixed with the perforated collar 9 to the outer tube 2. To disassemble the shown stem 1, the inner tube 3 is designed to be pulled in the proximal direction out of the outer tube 2.

One of the grooves 10 is shown in section in FIG. 2. FIGS. 1 and 2 show that the groove 10 runs in the axial direction of the stem 1. Its proximal end is rounded at 16 and its distal end is rounded at 17, the rounding and beveling of the groove ends precluding injury to the enclosing body tissue when longitudinally displacing the stem 1.

At its distal end constituted by the perforated collar 9, namely distally from the grooves 10, the outer tube comprises a distally closed end assuring high mechanical strength.

The proximal end of the flange 12 ends at the edge 18. As shown by FIGS. 1 and 2, the longitudinal edges of the groove 10, further the edge 18 and the proximal, rounded end 16 bound a distal aperture 19 through which the liquid can flow as indicated by the arrow 20 into the return flow duct 4. When the proximal zone of the groove 10 is overlapped by the urethra tightened against it, said liquid is unable to flow in the direction indicated by the arrow 20. However, farther distally, the liquid is able to enter, as indicated by the arrow 21 or 21, the groove 10, and therein it may flow underneath the enclosing body tissue covering the groove 10 through said groove's cross-section within the wall thickness of the outer tube 2 as far as the distal aperture 19.

The return flow duct 4 is cross-sectionally annular and is distally closed at 18, except at the distal apertures 19.

In the embodiment mode shown in FIGS. 1 and 2, the cross-section of the groove 10 allowing longitudinal flow is deepened by a recess 23 being constituted underneath said groove in the insulating muff 11, said recess in this embodiment mode and as a part 24 running as far as into the top side of the inner flange 12. The arrow 22 shows that the liquid also may flow in that region.

Return flow conditions are optimal when the grooves 10 are outward clear over their entire length. As indicated by the arrow 20, a flow of return liquid then may enter directly the passage 19 and next the return flow duct 4. When body tissue covers the groove 10 from the outside very far distally, whereby the liquid then can only enter the distal end of the groove 10 as indicated by the arrow 22. The return flow shall be slightly restricted, while return flow from a very distal stem zone still is possible.

FIGS. 4 through 7 show further embodiment modes of the stem 1 of the invention and are denoted as much as possible by the same references as used for the embodiment mode of FIGS. 1 through 3.

The embodiment mode of FIG. 4 substantially differs from that of FIG. 2 in that, the wall thickness of the outer tube 2 remains constant into the zone of the insulating muff 11 of lesser outside diameter. In this instance, the recesses 23 in the zone of the insulating muff 11 are absent. Also the inner flange 12 is absent. The inner tube 3 directly abuts by its enlarged end zone 15 the proximal edge 13 of the insulating muff 11.

In this embodiment mode of FIG. 4, the return flow conditions are similar to those of the embodiment of FIG. 2. If the groove 10 is outwardly clear over all its length, then the liquid is able to flow directly through the distal aperture 19 as indicated by the arrow 20 into the return flow duct 4. If groove 10 is covered over all its proximal zone, then the liquid shall flow as indicated by the arrow 22 at the distal end into the groove 10 and through same as far as the aperture 19.

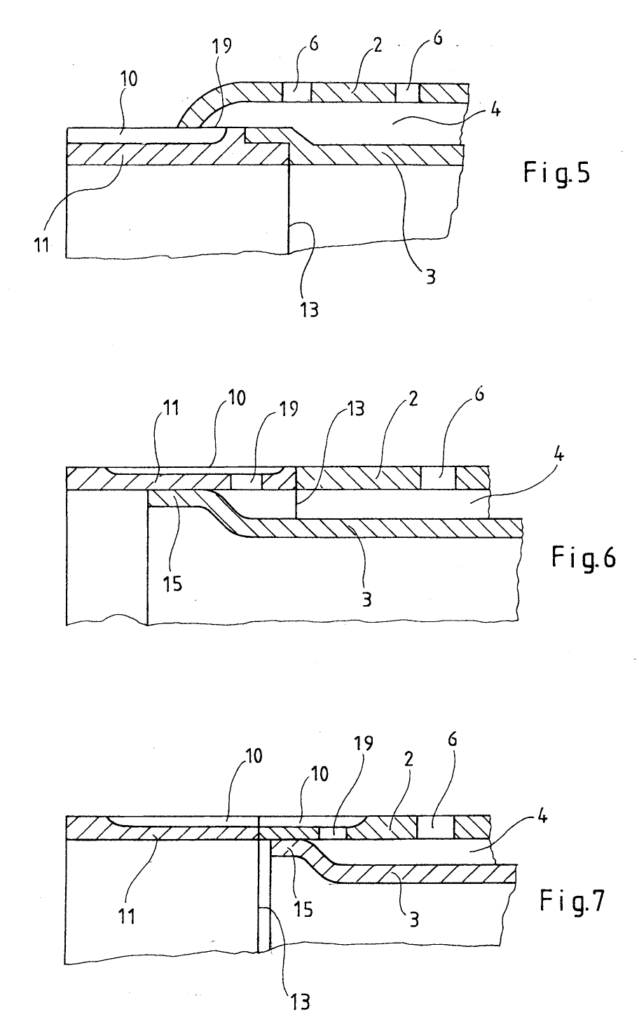

FIG. 5 shows a design variation wherein the insulating muff 11 is affixed by means of an overlap zone to the inner tube 3, The outer tube 2, fitted with apertures 6, externally overlaps the inner tube 3 and part of the insulating muff 11 and terminates by an inward-pointing front rim on the outer surface of the insulating muff 11 as shown in FIG. 5, The insulating muff 11 comprises grooves 10 which run in the distal direction and which in the present embodiment are open toward the distal edge of the insulating muff 11 and extend by their proximal end as far as underneath the outer tube 2, that is into the return flow duct 4, whereby they communicate there by means of the distal aperture 19 with the return flow duct 4.

As regards the embodiment mode shown in FIG. 6, the insulating muff 11 abuts the distal rim of the outer tube 2 to which it is joined for instance by soldering. The inner tube 3 is fitted with an enlarged distal end zone 15 of the inner tube 3 in the manner of the embodiment mode of FIG. 2, however it rests in an internally sealing manner in the embodiment mode of FIG. 6 against the insulating muff 11. In this latter embodiment as in that of FIG. 2, radial sealing is attained at the enlarged distal end zone 15 of the inner tube 3 and remains effective even when the inner tube 3 is displaced axially.

The embodiment mode of FIG. 6 again comprises grooves 10 fitted externally into the insulating muff 11 and communicating by means of distal apertures 19 with the return flow duct 4.

The embodiment of FIG. 7 is similar to that of FIG. 6. Again the insulating muff 11 abuts by its proximal edge 13 the distal rim of the outer tube 2 and illustratively is joined to it by soldering. On the other hand the inner tube 3 in the embodiment of FIG. 7 rests in a sealing manner by its enlarged end zone 15 against the outer tube 2. Accordingly the return flow duct still terminates within the outer tube 2.

In the embodiment mode of FIG. 7, the grooves 10 run distally and continuously both in the insulating muff 11 and in the outer tube 2 and communicate by distal apertures 19 with the return flow duct 4.

1. A urological resectoscope stem (1) comprising:
an insulating muff (11) constituting the distal end zone of said stem (1),
an outer tube (2, 9),
an inner tube (3), and
a cross-sectionally annular distally closed return flow duct (4) which is subtended at least over the larger part of its length between the outer tube (2, 9) and the inner tube (3), where the stem (1) is fitted with outwardly open, distally extending grooves (10), wherein the annular return flow duct (4) communicates directly by apertures (19) with the grooves (10).

2. The stem as claimed in claim 1, wherein the grooves (10) are made in the outer tube (2, 9).

3. The stem as claimed in claim 2, wherein the distal rim of the outer tube (2, 9) is closed.

4. The stem as claimed in claim 1, wherein the insulating muff (11) is overlapped, within a proximal zone of reduced diameter, by the outer tube (2, 9).

5. The stem as claimed in claim 4, wherein the grooves (10) are made in the outer tube (2,9) and the grooves (10) run distally as far as beyond the reduced zone of the insulating muff (11).

6. The stem as claimed in claim 5, wherein the grooves (10) are fitted with recesses (23) reaching as deep as into the insulating muff (11).

7. The stem as claimed in claim 1, wherein the outer tube (2, 9) is fitted with an inner flange (12) resting against the proximal edge (13) of the insulating muff (11), the inside diameter of said inner flange being widened in its proximal zone (14) up to the outside diameter of the inner tube (15).

8. The stem as claimed in claim 1, wherein the distal end zone of the outer tube (2) comprising the grooves (10) is a perforated collar (9) affixed to said outer tube.
